(19) United States
(12) Patent Application Publication
(10) Pub. No.: US 2008/0071328 A1
(43) Pub. Date: Mar. 20, 2008

(54) INITIATING MEDICAL SYSTEM COMMUNICATIONS

(75) Inventors: Gregory J. HAUBRICH, Champlin, MN (US); Charles H. DUDDING, Lino Lakes, MN (US); Christopher C. FULLER, Bloomington, MN (US)

Correspondence Address: INGRASSIA FISHER & LORENZ, P.C., 7150 E. CAMELBACK, STE. 325, SCOTTSDALE, AZ, 85251, US

(73) Assignee: MEDTRONIC, INC., MINNEAPOLIS, MN (US)

(21) Appl. No.: 11/850,206

(22) Filed: Sep. 5, 2007

Related U.S. Application Data
(60) Provisional application No. 60/824,687, filed on Sep. 6, 2006.

(51) Int. Cl. A61N 1/08

(52) U.S. Cl. 607/60

(57) ABSTRACT

A medical system includes a body-contacting signal source adapted to transmit an oscillatory signal though a body to a transducer of a device implanted therein. A detector that is coupled to the transducer, upon detection of a response of the transducer to the signal, activates a radio-frequency (RF) telemetry component of the device.

### CROSS-REFERENCE TO RELATED APPLICATION(S)

This application claims the benefit of U.S. provisional patent application Ser. No. 60/824,687, filed Sep. 6, 2006.

### TECHNICAL FIELD

Embodiments of the present invention pertain to medical systems including implantable devices and more particularly to initiating radio-frequency communications between devices of the medical systems.

### BACKGROUND

A wide variety of implantable medical devices (IMDs) are available for monitoring physiological conditions and/or delivering therapies. Examples of monitoring devices include, without limitation, hemodynamic monitors, ECG monitors, and glucose monitors. Examples of therapy delivery devices include, without limitation, electrical stimulation devices, such as cardiac pacemakers, cardioverter defibrillators, neurostimulators, and neuromuscular stimulators, and drug delivery devices, such as insulin pumps, morphine pumps, etc.

IMDs are often coupled to medical leads, extending from a housing enclosing the IMD circuitry. The leads carry sensors and/or electrodes and are used to dispose the sensors/electrodes at a targeted monitoring or therapy delivery site while providing electrical connection between the sensor/electrodes and the IMD circuitry. Leadless IMDs have also been described which incorporate electrodes and/or other types of sensors.

An integrated medical system tailored to a particular patient's medical needs may often include more than one of the aforementioned medical devices as well as one or more external devices that may provide a communications interface between a clinician and the implanted devices. A wireless communication network may be set up between the devices of the system in order to compile diagnostic data collected by one or more devices of the system and/or to coordinate effective therapy delivery among the devices. For example, therapy delivery devices of the system may be activated based on measurements, made by other devices of the system, and/or based on clinical analysis of measurements and/or responses to therapy delivery, reported by an external device of the system. However, if communications components in each device of the system were to remain active at all times, ready to receive communications from one another, a significant amount of power would be consumed. Thus, there is a need for a communications initiation mechanism, which can be incorporated into any or all of the implanted and external devices of the system, and is adapted to activate a communications component of any device within the system according to a demand for communications.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a conceptual diagram of a local communications network implemented in a medical system, according to some embodiments of the present invention.

FIG. 2 is a conceptual diagram illustrating a local communication network implemented within a mesh network architecture of a medical system.

FIG. 3 is a schematic diagram of an exemplary medical system having a local communications network that may incorporate one or more communication initiating mechanisms, according to some embodiments of the present invention.

FIG. 4 is a block diagram describing a functional relationship between implanted device components for communications initiation, according to some embodiments of the present invention.

FIG. 5 is a flow chart outlining some methods of the present invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Those skilled in the art will recognize that many of the examples provided have suitable alternatives that can be utilized. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

The present invention is directed to an ultra-low power, local communications network for use with a medical device system including one or more implanted devices. As used herein, the term “constellation” of devices refers to implantable medical devices deployed to targeted implant sites within signal-receiving range of an implanted or external pinging device, or signal source that transmits an activation signal. The term “distributed” medical devices refers to implantable devices that are implanted in a distributed manner throughout the patient's body, or a region of the patient's body, without being hardwired together by leads or other connectors. Medical devices included in a distributed medical device system will typically include leadless sensors and/or therapy delivery devices positioned at targeted monitoring/therapy delivery sites.

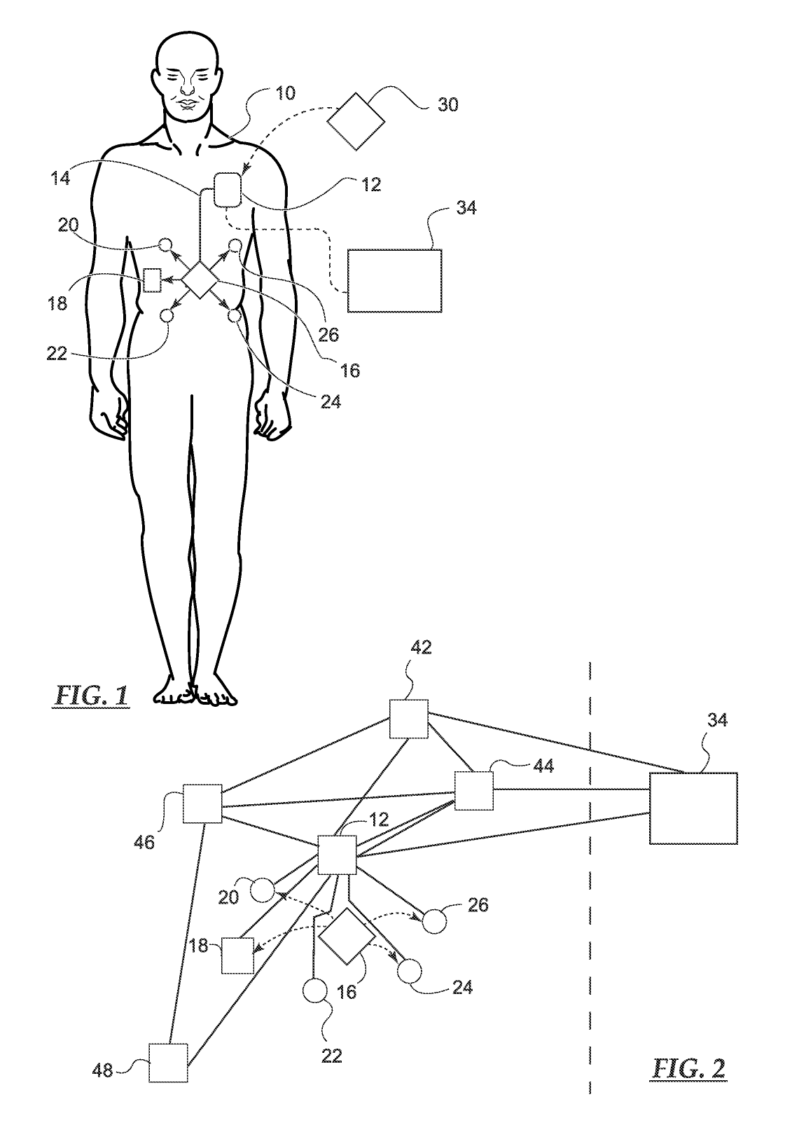

FIG. 1 is a conceptual diagram of a local communication network implemented in an implantable medical device system, according to some embodiments of the present invention. An IMD 12 is implanted in a patient 1O. IMD 12 is embodied as a cardiac stimulation device capable of delivering cardiac pacing, cardioverting and/or defibrillation therapies as well as sensing cardiac signals and optionally other physiological signals. IMD 12 may alternatively be embodied as any IMD capable of monitoring physiological signals and/or delivering therapy such as a neurostimulator, drug pump, hemodynamic monitor, or ECG monitor.

IMD 12 is shown coupled to a lead 14. Lead 14 carries one or more electrodes for sensing and/or delivering electrical stimulation therapies and may carry additional sensors for monitoring physiological signals. In other embodiments, IMD 12 may be coupled to multiple leads or alternatively be provided as a leadless device, incorporating electrodes and sensors on or in the housing of IMD 12. IMD 12 is enabled for bidirectional communication using RF telemetry or other wireless communication with an external device 34 such as a home monitor or programmer. One example of an appropriate RF telemetry communication system is generally described in commonly-assigned U.S. Pat. No. 6,482,154 (Haubrich, et al.), hereby incorporated herein by reference in its entirety.

Patient 10 is further implanted with a number of other devices 18, 20, 22, 24 and 26 disposed as a constellation of distributed devices. Device 18 may be a second therapy delivery device such as another electrical stimulation device or a drug pump. Devices 20, 22, 24 and 26 are embodied as implantable sensors and may include, but are not limited to, sensors for monitoring pressure, blood flow, acceleration, displacement, or blood/tissue chemistry such as oxygen saturation, carbon dioxide, pH, protein levels, enzyme levels, etc. Devices 12 through 26 represent a distributed system of implantable medical devices in that the devices are not coupled to each other by leads or conductors. Sensors 20 through 26 are implanted at targeted monitoring sites without limitations associated with lead-based sensors.

Devices 12 through 26 are provided with wireless communication connectivity in a local communications network. Devices 18 through 26 are arranged as a “constellation” or cluster of distributed devices within signal reception range of a local network pinging device 16. Local network pinging device 16 is shown coupled to lead 14. In other embodiments, pinging device 16 may also be embodied as a leadless device. Pinging device 16 may alternatively be incorporated in IMD 12 depending on the proximity of IMD 12 to the targeted constellation of devices 18 through 26 for successful receipt of and response to a wake-up signal generated by pinging device 16.

Device 18 and sensors 20 through 26 include a power source, which may be a stand-alone battery, a rechargeable storage device such as a rechargeable battery or capacitor (which may be recharged internally or transcutaneously with the use of electromagnetic or piezoelectric transformers), or an energy-harvesting device. Device 18 and sensors 20 through 26 further include a physiological sensor (which is optional in therapy delivery device 18) and a processor and associated memory for controlling device communication functions and storing data as needed. Device 18 and sensors 20 through 26 are provided with an RF telemetry transmitter or transceiver to allow devices 18 through 26 to transmit data to IMD 12 and/or external device 34.

Device 18 and sensors 20 through 26 are normally in an ultra-low power “OFF,” state and are responsive to an acoustic or RF ping signal generated by pinging device 16. During the OFF state, no active circuitry is consuming power, such that the only energy consumed by the device is due to leakage currents, which are generally in the nA range. No power is consumed by the data communications circuitry, and power control circuitry essentially opens the power supply lines to all power-dependent device circuitry or modules. The power control circuitry is in an OFF state as well.

Pinging device 16 generates a ping signal on a scheduled or manually or automatically triggered basis. The ping signal causes a ping detector included in device 18 and sensors 20 through 26 to wake-up power control circuitry which then wakes up the microprocessor included in device 18 and sensors 20 through 26 thus transitioning device 18 and sensors 20 through 26 to a high power “ON” state. The microprocessor subsequently wakes up communications circuitry. This transition to a high-power “ON” state enables the telemetry circuitry of device 18 and sensors 20 through 26 for receiving commands or requests via an RF communication link in a bidirectional operation mode or for transmitting data in a transmit-only mode. The wake-up response to a ping signal may be based on charge accumulation reaching a wake-up threshold or based on a resonance response to an incident frequency. In one embodiment, the ping detector is an acoustic sensor or transducer which turns on a switch which powers up a bootstrap circuit to take the control and microprocessor circuitry out of an ultra-low power OFF state to a high-power ON state. In an alternative embodiment, the ping detector includes an RF energy detector, e.g., a resonant circuit in RFID or Tag systems) and the energy coupled to the ping detector causes a switch to close subsequently resulting in a powering up of the power control circuitry, microprocessor, communication circuitry and other device components. Other mechanisms for wake-up responses of devices 18 through 26 to a ping signal may be implemented. The response of an acoustic or RF ping detector is rapid allowing minimal latency between generation of a ping signal and initiation of the powering up. Thus the response time of the overall system can be minimized to allow a rapid response of the system to changing conditions.

Upon receiving the wake-up signal from pinging device 16, device 18 and sensors 20 through 26 commence an RF data communication session for transmitting and/or receiving data from IMD 12 and/or an external device 34. Sensors 20 through 26 may be embodied as transmit-only devices for sending data through an RF communication link to IMD 12 or external device 34 using an Aloha supervised communication scheme with redundancy or other communication protocol for reducing data packet collisions. For example, data transmissions may be staggered through time using different time delay signals for each addressed device. If autonomous supervision of data transmission is not implemented, the power consumption of sensors 20 through 26 operating in a transmit-only mode can be extremely low with power being consumed only when a sensor is actively pinged. The longevity of the implanted sensors 20 through 26 may approach the self-discharge rate of the sensor power source.

Sensors 20 through 26 may alternatively be enabled for bi-directional communication and may alternate between transmit-only and bidirectional communication modes depending on the power status of the sensor, the operational workload of the sensor for monitoring physiological signals, and the status of the patient. Device 18 will typically be enabled for bi-directional communication but may also be embodied with transmit-only capabilities.

In past practice, an implanted device is programmed to “wake-up” at prescheduled times or remains in a low-power but “alert” state for receiving communication requests. By providing a pinging device 16 for waking up the devices 18 through 26 from an “OFF” state, communication sessions can be initiated at any time without waiting for a scheduled wake-up of devices 18 through 26. The power consumption burden normally required for maintaining devices 18 through 26 in a low-power “alert” state is reduced by allowing devices 18 through 26 to remain in an even lower power OFF state until actively pinged. By reducing the power required for enabling local communication connectivity in the implanted system, the overall size of each of the constellation devices 18 through 26 can be reduced.

Pinging device 16 can be implemented as a simple beacon device for waking up all implanted devices 18 through 26. Alternatively, pinging device 16 may be enabled to address individual devices or groups of devices through implementation of an addressing scheme based on frequency, time or digital code.

Device 18 and sensors 20 through 26 may operate in a variety of modes depending on clinician preference and patient condition. For example, device 18 and sensors 20 through 26 may be in an “OFF” state until awoken by pinging device 16 after which the addressed devices are turned “ON” and commence device functions which may include sensing, data processing, therapy delivery, data transmission, or receiving data requests, programming instructions or other data/commands. In other embodiments, device 18 and sensors 20 through 26 may be operating in a low-level state carrying out basic device functions, such as continuous or periodic monitoring of a physiological signal with data storage, and upon receiving a “wake-up” signal from pinging device 16, convert to a high power state for carrying out additional operations such as data processing and/or data communications. Some devices included in the constellation of distributed devices may be used only at specific times such as during therapy adjustments (e.g., during reprogramming of IMD 12 or during changes in medications or drug dosages). As such, implanted devices 18 through 26 may be available any time a clinician would like to collect additional data or information about the patient's status, remaining in an “OFF” state until actively turned “ON” by pinging device 16.

When pinging device 16 is coupled to IMD 12 by lead 14, pinging device 16 may receive power from conductors extending through lead 14 to the power supply of IMD 12 and receive signals from IMD 12 via conductors extending through lead 14 for triggering pinging device 16 to issue a ping or wake-up signal to one or more of device 18 and sensors 20 through 26. Alternatively, pinging device 16 may be embodied as leadless device having its own power supply (a stand alone battery, rechargeable battery or capacitor, or energy-harvesting device) and enabled for receiving RF telemetry signals from IMD 12 and/or external device 34 for triggering generation of a ping signal. As such, pinging device 16 includes a power supply, a communication link with IMD 12 (which may be wireless or hardwired), and/or a communication link with external device 34 and a signal generator for emitting a ping signal, which may be an acoustical or RF signal, to wake up device 18 and sensors 20 through 26. Pinging device 16 may include a processor and associated memory for controlling the generation of ping signals addressed to specific devices and may operate supervisory protocols for ensuring reliable RF data transmission. Only pinging device 16 need remain in a low-power alert state for receiving communication requests from IMD 12 and/or external device 34, thereby allowing the constellation of distributed devices 18 through 26 to remain in an ultra-low power OFF state.

A local communications network including pinging device 16 may change in membership at any time when new devices are implanted or when existing devices are functionally depleted or physically removed. As such, the constellation of implanted devices can expand “organically” as new sensor and therapy delivery devices are implanted for monitoring and managing a patient's disease progress.

Each of device 18 and sensors 20 through 26 may further be enabled for bidirectional communication with external device 34 to allow for programming of operating modes and control parameters and for transmitting data acquired by the implanted devices 18 through 26 to external device 34. External device 34 may accumulate, prioritize and transfer data as appropriate for notifying the patient 10, a caregiver, a clinician, a clinical database, emergency responders or other external device or communications network of a patient condition, physiological event, or device status. Reference is made to commonly-assigned U.S. Pat. No. 6,599,250 (Webb et al.), U.S. Pat. No. 6,442,433 (Linberg et al.) U.S. Pat. No. 6,622,045 (Snell et al.), U.S. Pat. No. 6,418,346 (Nelson et al.), and U.S. Pat. No. 6,480,745 (Nelson et al.) for general descriptions of examples of network communication systems for use with implantable medical devices for remote patient monitoring and device programming, all of which are hereby incorporated herein by reference in their entirety.

In addition to responding to a ping signal, device 18 and sensors 20 through 26 may be pre-programmed to autonomously wake up and perform sensing, data communication, and other functions at scheduled intervals with data transmitted to IMD 12 and/or external device 34. It is further contemplated that in an awake mode, device 18 and sensors 20 through 26 may communicate with each other in either transmit-only or bidirectional communication modes. RF communication links made available through the implantable medical device system, including both implanted devices and external devices, may be implemented according to the particular application, clinician preference, and individual patient need.

RF communications may be executed between devices 18 through 26 and IMD 12 and/or external device 34 on any selected operating frequency bands such as MICS, MEDS, and ISM. If data from any of the addressed devices is not received by the IMD 12 and/or external device 34 within an expected time window subsequent to generation of the ping signal, the constellation of devices 18 through 26 may be collectively or selectively re-pinged. Repeated attempts may be made according to data priority and communication rules in place, which may be stored in the memory of IMD 12 or pinging device 16.

In some embodiments, a patient may be implanted with a constellation of distributed sensors 20 through 26 for collecting physiological data for diagnostic or patient monitoring purposes without being implanted with a therapy delivery device such as IMD 12. Pinging device 16 operates to wake-up sensors 20 through 26 to initiate data communications and may also receive RF transmitted data from sensors 20 through 26 for storage and transfer to an external device 34. Alternatively or additionally, an external pinging device 30 may be provided which can wake up sensors 20 through 26 to initiate communication operations between sensors 20 through 26 and external device 34. When IMD 12 is present, IMD 12 may also be responsive to an externally generated ping signal from external pinging device 30. External pinging device 30 may be implemented as a stand-alone device that may be manually triggered by a user, such as a patient, caregiver, clinician, or emergency responder. Alternatively, external pinging device 30 may be embodied in external hospital monitoring equipment, an automatic external defibrillator (AED), an external home monitor 34, or a patient activator or other handheld device.

FIG. 2 is a conceptual diagram illustrating a local communication network implemented within a mesh network architecture of an implantable medical device system. IMD 12 may be implemented as a network member (node) of a mesh architecture implantable medical device communication system, as generally described in co-pending U.S. patent application Ser. No. 11/739,388. IMD 12 is shown to be networked with multiple implantable devices 42, 44, 46 and 48 and with external device 34. Each of devices 12, 42, 44, 46, 48, and 34 function as nodes of the mesh network allowing multi-hop data transmissions between devices 12, 42, 44, 46, 48, and 34. Each device is enabled to communicate wirelessly along multiple pathways with each of the other networked devices. Only examples of some of the shorter communication pathways are shown in FIG. 2 for the sake of simplicity. The mesh network is a self-configuring, self-healing network responsive to changes in network membership, changes in patient condition, and changes in the individual power status of network members. Implanted networked devices 42, 44, 46 and 48 may include specialized nodes assigned to perform network tasks such as data processing, data storage, gateway, scheduling, etc. Devices 42 through 48 may further include physiological sensing and/or therapy delivery functions.

IMD 12 is configured to receive data packets from the local constellation of device 18 and sensors 20 through 26 responsive to ping signals received from pinging device 16. IMD 12 may then transmit data received from the local constellation of devices 18 through 26 to any of the networked implanted devices 42 through 48 and external device 34 according to a channel plan and routing scheme currently effective in the mesh network. As such data collected by IMD 12 from the local constellation of devices 18 through 26 may be used directly by IMD 12 or transmitted to another device included in the implanted system via the mesh network for use by the other device.

It is contemplated that, according to some embodiments of the present invention, an individual patient may be implanted with multiple constellations of distributed medical devices, each including a ping device. Each constellation of devices would be disposed within signal-receiving distance from a pinging device for that constellation. When multiple pinging devices are implanted, only one needs to remain in a low-power alert state for receiving a communication request from an IMD or external device. The alert pinging device would then emit a ping signal to “wake-up” the remainder of the pinging devices which would each, in turn, emit pinging signals to their respective constellation of devices. As such each pinging device may also be configured with a processor responsive to a ping signal. The duty of operating as a “wake-up master” could be transferred to different pinging devices based on individual pinging device power status or other patient-related priorities.

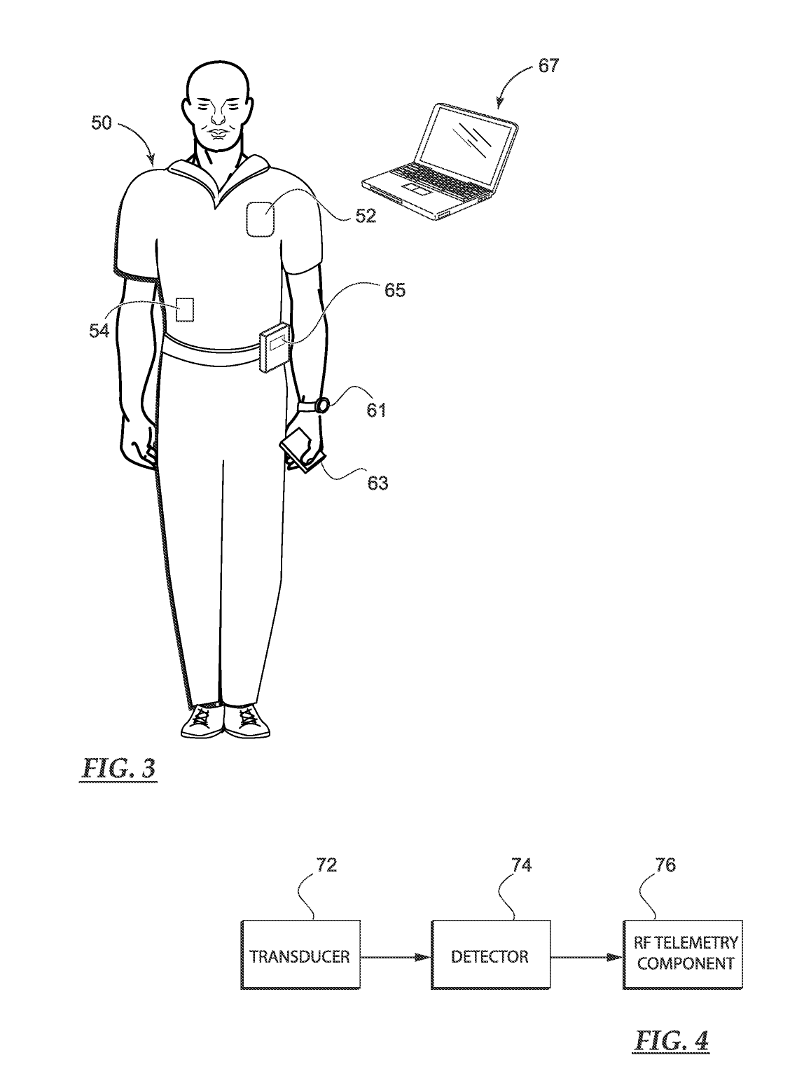

FIG. 3 is a schematic diagram of an exemplary medical system having a local communications network that may incorporate one or more communication initiating mechanisms, according to some embodiments of the present invention. FIG. 3 illustrates a patient 50 in whose body a first implantable medical device 52 and a second implantable medical device 54 are implanted. FIG. 3 further illustrates patient 50 wearing a first external device 61 around a wrist, wearing a second external device 65 around a waist, and holding a third external device 63. Any one, or all, of external devices 61, 63, 65, along with a device analyzer/programmer 67, such as is known to those skilled in the art, may be included in the exemplary medical system. According to the illustrated embodiment, at least one of implanted devices 52 and 54 includes a communications module, including an RF telemetry component 76 (FIG. 4), to enable communication via RF telemetry; component 76 may be either a transmitter, a receiver, or a transceiver, which is activated, via a signal sent from a signal source, which may be included in any one of devices 52, 54, 61, 63, 65 and 67, in order to initiate communications. FIG. 4 is a block diagram describing a functional relationship between implanted device components for communications initiation, according to some embodiments of the present invention. FIG. 4 illustrates a transducer 72, for receiving the activation signal, coupled to a detector 74; upon detection of a response of transducer 72 to the signal, which may be amplified, detector 74 activates telemetry component 76 to initiate communications. According to embodiments of the present invention, the communication module remains in an ultra-low power “OFF” state until telemetry component 76 is activated.

According to preferred embodiments of the present invention, the signal source corresponds to any of the previously described embodiments of pinging device 16, 30. The signal source transmits an oscillatory signal, in particular an ultrasound signal, for example, having a frequency greater than approximately 20 kHz, which is received by an acoustic type of transducer 72. According to alternate embodiments, the signal source is optical in nature, transmitting an infrared signal, for example, being in the frequency range between approximately 4.3×10¹⁴ Hz and approximately 5.0×10¹⁴ Hz, to be received by an optical type of transducer 72, for example a photo-detector. As previously described for ultrasonic ping detection, the response to either the acoustic or optical activation signals is relatively rapid for minimal latency between generation of the signal and initiation of communications.

With reference to FIG. 3, any of devices 52, 54, 61, 63, 65 may include a signal source, or pinging device, to transmit an acoustic activation signal to the acoustic-type of transducer 72, included in either of implanted devices 52, 54, however, only device 63, shown held in the hand of patient 50, may be able to transmit an optical activation signal to either of implanted devices 54, 52. Optical signal transmission through the body of patient 50 will require a relatively close alignment between the signal source, for example a light emitting diode (LED), and the optical type of transducer 72; the optical signal may be transmitted to transducer 72, for example, in the form of a photo-detector, included in either device 52 or 54, by holding device 63 in close contact with a surface of the body of patient 50 beneath which device 52 or 54 is implanted. Of course, devices 52 and 54 may have been implanted in closer proximity to one another, with optical signal transmission in mind, so that transmission of an optical activation signal from one to another may be enabled.

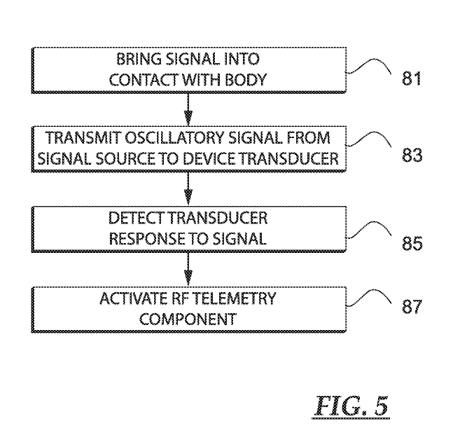

FIG. 5 is a flow chart outlining some methods of the present invention for initiating communications with a medical device. According to the FIG. 5 flow chart, an initial step 81 for initiating communications with a medical device is to bring the signal source into contact with the body in which the medical device is implanted. If the signal source is included in another implanted device, step 81 will have been performed at the time the device including the signal source is implanted, which may have been just prior to, coincident with, or after the medical device was implanted. Otherwise, step 81 is performed by bringing an external device including the signal source into contact with an external surface of the body. Once in contact with the body, an oscillatory signal, for example, ultrasonic or infrared, is transmitted from the signal source to the device transducer, per step 83, with the intent of activating the RF telemetry component, per step 87, via detection of the transducer response to the signal, per step 85. Step 83 may be performed in response to a condition detected by one or more sensors of the device that includes the signal source, or in response to a predetermined communications schedule.

Referring back to FIG. 3, in conjunction with FIG. 5, various embodiments of a medical system, operating according to steps of FIG. 5, will now be described, being categorized into several groups. It should be noted that additional system embodiments formed by combinations of embodiments from the groups described below are within the scope of the present invention.

According to a first group of embodiments, device 52 has the capacity to deliver therapy to the body of patient 50, based on sensed conditions, and may have additional capacity to sense one or more conditions of the body of patient 50; and device 54 has only the capacity to sense one or more conditions of the body. According some embodiments of this first group, device 54 includes the signal source, or pinging device, which transmits the activation signal, to initiate communications with device 52, upon detection by device 54 of a condition for which related information should be transferred to device 52. The information may be processed in device 52, to aid in a selection of an appropriate therapy to be delivered from device 52, or the information may be transferred from device 52, via the activated RF telemetry component of device 52, out to an external device, for example, any of devices 61, 63, 65, 67, to ultimately inform and/or warn patient 50 and/or a clinician of the condition. Information transferred to the external device may be related to a functional condition of patient 50 or device 54 itself, for example, a failure or impending failure of a component of device 54. According to alternate embodiments of this first group, device 52 has the additional capacity to sense one or more conditions and includes the signal source, which sends the activation signal to initiate communications with device 54, when information from device 54, based on the condition(s) sensed by device 54, is required in order to augment the information based on the condition(s) sensed by device 52, so that device 52 may decide whether or not to proceed with a therapy.

According to a second group of embodiments, an external body-worn device, for example, device 61 or 65, or a hand-held device, for example, device 63, includes the signal source for transmitting the signal to initiate communications with one or more implanted devices, for example, devices 52, 54, and/or to initiate communications between a plurality of implanted devices. According to some embodiments of this second group, the external device is pre-programmed, or manually activated, via an external interface of the device, to transmit the activation signal according to a predetermined schedule for interrogation and/or programming of the implanted device(s), which may be performed by any of body contacting external devices 61, 63, 65, or by another external device, for example, analyzer/programmer 67. Any of devices 61, 63 and 65 may include a display for communicating messages received from the implanted device(s) once RF telemetry communications have been initiated, as well as capacity to store and/or analyze data transferred from the implanted device(s). Any of external devices 61, 63, 65 may further include the capacity to program either or both of implanted devices 52, 54, via the activated RF telemetry communications.

In the foregoing detailed description, the invention has been described with reference to specific embodiments. However, it may be appreciated that various modifications and changes can be made without departing from the scope of the invention as set forth in the appended claims.

What is claimed is:

1. A method for initiating communications with a medical device, the method comprising:
bringing a signal source into contact with a body;
transmitting an oscillatory signal through the body, in which the medical device is implanted, from the signal source to a transducer being coupled to an RF telemetry component of the medical device;
detecting a response of the transducer to the signal; and
activating the RF telemetry component upon detection of the response in order to initiate communications.

2. The method of claim 1, wherein the step of bringing the signal source into contact with the body comprises implanting a device containing the signal source into the body.

3. The method of claim 1, wherein the step of bringing the signal source into contact with the body comprises holding a device containing the signal source against an external surface of the body.

4. The method of claim 1, wherein the step of transmitting is in response to a condition detected by a device containing the signal source.

5. The method of claim 1, wherein the step of transmitting is in response to a predetermined schedule.

6. The method of claim 1, wherein the oscillatory signal is ultrasonic.

7. The method of claim 1, wherein the oscillatory signal is infrared.

8. A medical system, comprising:
at least one implantable device including an RF telemetry component coupled to a signal detector, and a signal transducer coupled to the signal detector, the signal detector controlling activation of the RF telemetry component;
a body-contacting signal source adapted to transmit an oscillatory signal through a body to the transducer of the device, when the device is implanted in the body;
the signal detector, upon detection of a response of the transducer to the signal, activating the RF telemetry component to initiate communications with the implanted device.

9. The medical system of claim 8, wherein the telemetry component is a receiver only.

10. The medical system of claim 8, wherein the telemetry component is a transmitter only.

11. The medical system of claim 8, wherein the telemetry component is a transceiver.

12. The medical system of claim 8, wherein the signal source and the signal transducer are acoustic.

13. The medical system of claim 8, wherein the signal source and the signal transducer are optical.

14. The medical system of claim 8, wherein the signal source is coupled to an implantable sensor, the sensor adapted to control transmission of the signal from the source to the transducer based on a condition sensed by the sensor, when the sensor is implanted in the body.

15. The medical system of claim 14, wherein:
the condition is related to a functional aspect of the body; and
the implanted device is adapted to deliver a therapy according to information received via the communications initiated by the activation of the RF telemetry component by the transducer.

16. The system of claim 14, wherein:
the condition is related to a functional aspect of the sensor; and
the implanted device is adapted to deliver a warning according to information received via the communications initiated by the activation of the RF telemetry component by the transducer.

17. The medical system of claim 8, wherein:
the implantable device is a sensor; and
the signal source is coupled to an implantable therapy delivery device, the therapy delivery device, when implanted, adapted to sense conditions, to control transmission of the signal to the transducer based on the sensed conditions, and to deliver or withhold a therapy according to the sensed conditions in combination with information received from the implanted sensor via the communications initiated by the activation of the RF telemetry component by the transducer.

18. The medical system of claim 8, wherein the signal source is coupled to a body-worn device, including an external interface adapted for controlling the signal source.

19. The medical system of claim 18, wherein the external interface of the body-worn device is further adapted to display messages pertaining to the communications initiated with the implanted device.

20. An implantable medical device, comprising:
a communications module for RF telemetry, the module including a telemetry component being one of a receiver, a transmitter, and a transceiver; and
an acoustic transducer coupled to a detector, the detector coupled to the telemetry component, the detector being adapted to activate the telemetry component upon detection of a response of the transducer to an acoustic activation signal.
